(19) United States
(12) Patent Application Publication
(10) Pub. No.: US 2018/0116301 A1
(43) Pub. Date: May 3, 2018

(54) LOWER LUMBAR SUPPORT LEGGING

(71) Applicant: Eric Shin, Montebello, CA (US)

(72) Inventor: Eric Shin, Montebello, CA (US)

(21) Appl. No.: 15/339,725

(22) Filed: Oct. 31, 2016

(51) Int. Cl. A41D 1/08; A41D 31/00; A41F 9/00; A61F 5/02

(52) U.S. Cl. CPC A41D 1/08; A41D 31/00; A41D 2400/22; A61F 5/028; A41F 9/00

(57) ABSTRACT

Lower lumbar leggings may be worn in connection with various activities, including work, exercise, physical therapy, everyday leisure, and the like. The legging can have an open-ended torso section for extending about a user's lower lumbar region. At least a portion of the front of the open-ended torso section can include an elastic portion, while at least a portion of the back of the open-ended torso section can include a non-stretch fabric designed to help maintain the lower back in a proper position. One or more straps can extend from this non-stretch back portion and may be stretched to attach at front sides of the open-ended torso section. The leggings may be designed as full-length leggings, capris, shorts, or the like, as the specific length of the leg portions of the leggings does not affect the benefits of the lower lumbar support provided by the upper portion thereof.

### BACKGROUND OF THE INVENTION

### 1. Field of the Invention

One or more embodiments of the invention relates generally to leggings. More particularly, the invention relates to a lower lumbar legging that includes a strap portion that may be adjustably tightened to provide lower lumbar support.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Posture refers to the position of a person's body when standing or sitting. It is a result of reflexes, habits, and adaptive responses to anything that resists a person being more or less upright and functional. Posture can be affected by gravity, working conditions, anatomy, hobbies or athletics, and the like.

Poor posture is the posture that results from certain muscles tightening up or shortening while others lengthen and become weak which often occurs as a result of one's daily activities. After a time, poor posture feels normal and continues to regress further from correct posture. When poor posture feels normal, it becomes harder to correct because the muscle memory now stores the information needed for poor posture. Poor posture will continue and increase one's digression from normal posture the longer it is left uncorrected.

There are numerous risks associated with poor posture. If not addressed properly, one's posture can be further harmed and can lead to increasingly painful experiences. Any distress in the spine, as well as other parts of the body can be increased due to prolonged periods of poor posture. Poor posture can impede the ability of the lungs to expand. Posture, when correct, helps to increases one's ability to breathe, and allows muscles to work at optimum capacity.

Many products are available to address poor posture. Some of these devices cannot be worn for various tasks and some of these devices have been found to do more harm than good. Being aware of their posture, straightening their back, lifting their chest, rolling their shoulders back and rotating their pelvis so their stomach and behind are tucked in will help improve one's posture. However, unless posture is part of a person's sport or exercise, people will most likely have a hard time being consistent with these physical posture correcting exercises.

As can be seen, there is a need for a device to help correct a person's posture and provide lumbar spine support that is safe and can be effective, even when the person is performing various typical daily tasks.

### SUMMARY OF THE INVENTION

Embodiments of the present invention provide lumbar support leggings comprising leg openings for receiving a user's legs; and an open-ended torso region formed integrally with the leg openings, the open-ended torso region fitting about at least a portion of a user's torso, the open-ended torso region having a front panel formed from a flexible material, hook receiving fabric attached at each side of the front panel, a non-stretch fabric region configured to be disposed adjacent a user's lower lumber spine, and at least one strap extending from each side of the non-stretch fabric region, each strap configured to removably attach to respective hook receiving fabric at each side of the open-ended torso region.

Embodiments of the present invention further provide lumbar support leggings comprising leg openings for receiving a user's legs; and an open-ended torso region formed integrally with the leg openings, the open-ended torso region fitting about at least a portion of a user's torso, the open-ended torso region having a front panel formed from a flexible material, hook receiving fabric attached at each side of the front panel, a non-stretch fabric region configured to be disposed adjacent a user's lower lumber spine, and a upper elastic strap and a lower elastic strap extending from each side of the non-stretch fabric region, each strap configured to removably attach to respective hook receiving fabric at each side of the open-ended torso region.

Embodiments of the present invention also provide a method for supporting a lower lumbar back of a user comprising donning lumbar support leggings, the lumbar support leggings having leg openings receiving a user's legs and an open-ended torso region formed integrally with the leg openings, the open-ended torso region fitted about at least a portion of a user's torso, the open-ended torso region having a front panel formed from a flexible material, hook receiving fabric attached at each side of the front panel, a non-stretch fabric region configured to be disposed adjacent a user's lower lumber spine, and at least one strap extending from each side of the non-stretch fabric region, each strap configured to removably attach to respective hook receiving fabric at each side of the open-ended torso region; pulling on each of the straps to cause the non-stretch fabric region to apply a selected pressure to the lower lumber back of the user; and securing the straps to the hook receiving fabric to maintain the selected pressure.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 is a front view of a lower lumbar legging, illustrating attached straps, according to an exemplary embodiment of the present invention;

FIG. 2 is a back view of the lower lumbar legging of FIG. 1, illustrating the straps attached;

FIG. 3 is a front view of the lower lumbar legging of FIG. 1, illustrating the straps unattached; and

FIG. 4 is a back view of the lower lumbar lagging of FIG. 1, illustrating the straps unattached.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a lower lumbar legging that may be worn in connection with various activities, including work, exercise, physical therapy, everyday leisure, and the like. The legging can have an open-ended torso section for extending about a user's lower lumbar region. At least a portion of the front of the open-ended torso section can include an elastic portion, while at least a portion of the back of the open-ended torso section can include a non-stretch fabric designed to help maintain the lower back in a proper position. One or more straps can extend from this non-stretch back portion and may be stretched to attach at front sides of the open-ended torso section. The leggings may be designed as full-length leggings, capris, shorts, or the like, as the specific length of the leg portions of the leggings does not affect the benefits of the lower lumbar support provided by the upper portion thereof.

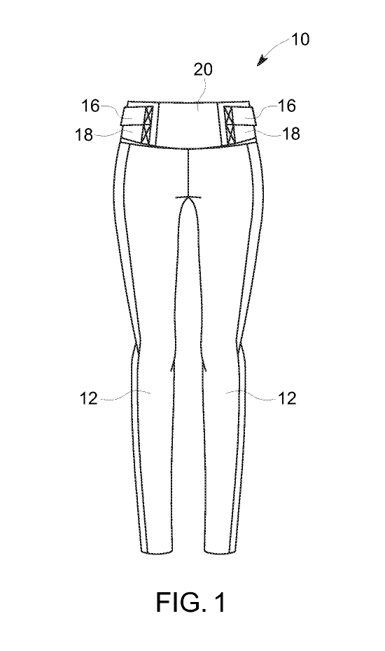

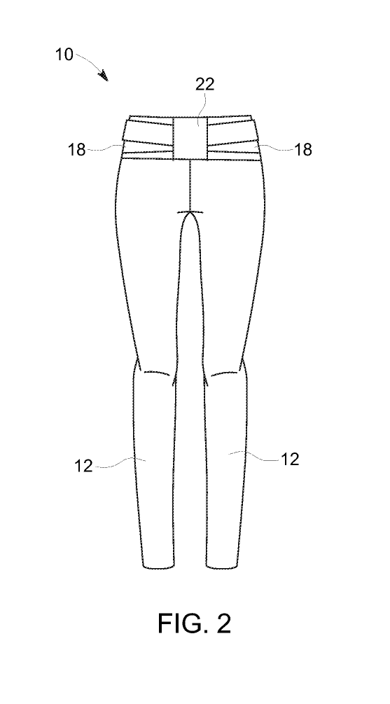

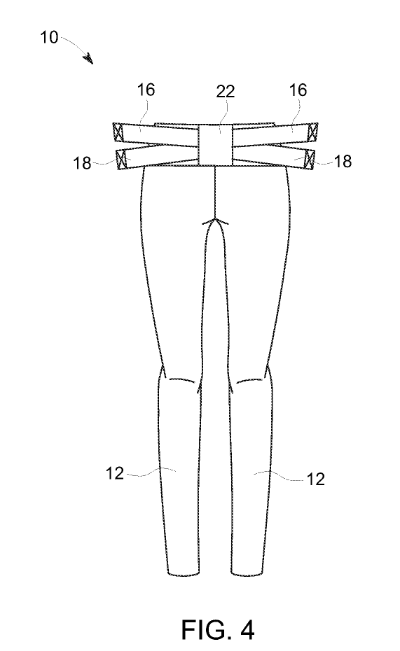

Referring now to FIGS. 1 through 4, lower lumber leggings 10, also referred to simply as leggings 10, can include leg openings 12, or simply, legs 12, extending from an open-ended torso section. As discussed above, the legs 12 can be of various length, creating leggings 10 that may be configured as underpants, shorts, capris, pants, or the like. The legs 12 can be formed from various materials and may be made to be loose or form-fitting. In some embodiments, the legs 12 may be made from fabrics such as polyester blends, nylon blends and fabrics that wick moisture from the skin to create a dry touch.

The open-ended torso region may include a front central panel 20 formed from a flexible fabric which may be the same or different of the fabric of the legs 12. Hook receiving fabric 14 may be disposed adjacent and on each side of the front central panel 20. The hook receiving fabric 14 may be a stretchable or non-stretchable fabric, for example, suitable for receiving a hook portion of a hook and loop type fastener.

The hook receiving fabric 14 may form the remainder of the waistband (the open-ended torso section) with a non-stretch fabric region 22 at the back center of the open-ended torso section of the leggings 10. The non-stretch fabric region 22 may be made from various materials and may be perforated for breathability, for example.

In some embodiments, the hook receiving fabric 14 may extend to and be connected with the non-stretch fabric region 22. In other embodiments, an intermediate fabric portion may be disposed between the hook receiving fabric 14 and the non-stretch fabric region 22, as hook receiving material may not be required along the back portion of the sides of the leggings 10.

One or more straps may extend from each side of the non-stretch fabric region 22 and extend about a portion of the user's torso. In some embodiments, as shown in the Figures, an upper strap 16 and a lower strap 18 may extend from each side of the non-stretch fabric region 22. The straps 16, 18 may partially overlap, as shown in FIG. 1, for example, when sewn or attached to the non-stretch fabric region 22 for extra support of the lower back. In some embodiments, the straps 16, 18 may be formed from an elastic material.

The straps 16, 18 may have hook portions 24 having hook material for engagement with the hook receiving fabric 14. The hook portions 24, having the hook material, while shown just on the last portion of each of the straps 16, 18, may be of various proportions of the total length of the straps 16, 18. In some embodiments, the hook portions 24 may cover some or all of one side (a user-facing side) of the straps 16, 18.

The open-ended torso section of the leggings 10 can be formed in various heights. In other words, the amount that the leggings 10 will come up the user's back may vary. Typically, the height of the non-stretch fabric region 22 may be from about 3 to about 8 inches, with each strap 16, 18 being from about 0.75 to about 3 inches wide, for example.

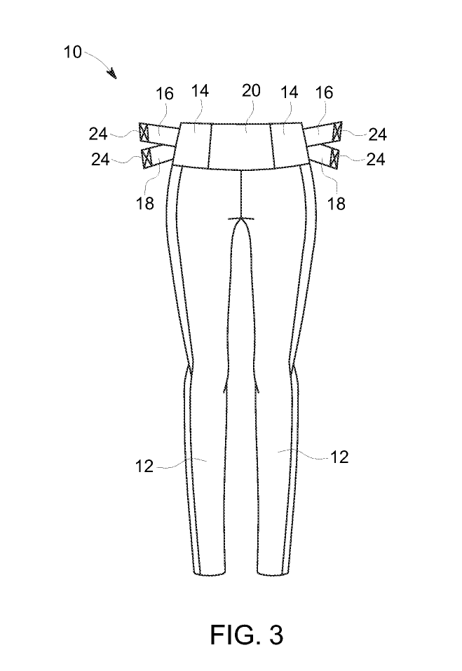

To use, a user may simply apply the leggings 10 similar to a pair of pants, with the non-stretch fabric region 22 positioned at the user's lower lumber spine. The initial placement of the leggings 10 may be made with the straps 16, 18 disconnected, as shown in FIG. 3. Once positioned, the straps 16, 18 may be elastically extended and the hook portions 24 of the straps 16, 18 secured to the hook receiving fabric 14. The amount of force applied to the non-stretch fabric region 22 may vary depending on how much the straps 16, 18 are elastically extended before being secured to the hook receiving fabric 14. Thus, the user may be able to control the amount of force exerted on their lower lumber region.

Use of the leggings 10 may, for example, help alleviate minor to moderate back pain or may aid in the healing after working out or doing strenuous activities.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. Lumbar support leggings comprising:
leg openings for receiving a user's legs;
an open-ended torso region formed integrally with the leg openings, the open-ended torso region fitting about at least a portion of a user's torso, the open-ended torso region having:
a front panel formed from a flexible material with at least one side;
a non-stretch fabric region configured to be disposed adjacent a user's lower lumber spine; and
at least one strap extending from each side of the non-stretch fabric region, each strap configured to removably attach to each side of the open-ended torso region.

2. The lumbar support leggings of claim 1, wherein said at least one side of the front panel has hook receiving fabric and said strap attached to the non-stretch fabric region removably attaches to said hook receiving fabric.

3. The lumbar support leggings of claim 2, wherein the at least one strap is at least two straps extending from each side of the non-stretch fabric region.

4. The lumbar support leggings of claim 3, wherein the at least two straps overlap at least where the straps are attached to the non-stretch fabric region.

5. The lumbar support leggings of claim 1, wherein the at least one strap is formed from an elastic material.

6. The lumbar support leggings of claim 1, wherein the non-stretch fabric region is formed from a perforated fabric.

7. The lumbar support leggings of claim 1, wherein the leg openings include leg fabric extending therefrom to form legs of the leggings.

8. Lumbar support leggings comprising:
leg openings for receiving a user's legs;
an open-ended torso region formed integrally with the leg openings, the open-ended torso region fitting about at least a portion of a user's torso, the open-ended torso region having:
a front panel formed from a flexible material with at least one side;
a non-stretch fabric region configured to be disposed adjacent a user's lower lumber spine; and
an upper elastic strap and a lower elastic strap extending from each side of the non-stretch fabric region, each strap configured to removably attach to respective hook receiving fabric at each side of the open-ended torso region.)

9. The lumbar support leggings of claim 8, wherein said at least one side of the front panel has hook receiving fabric and said strap attached to the non-stretch fabric region removably attaches to said hook receiving fabric.

10. The lumbar support leggings of claim 8, wherein the upper and lower straps overlap at least where the upper and lower straps are attached to the non-stretch fabric region.

11. The lumbar support leggings of claim 8, wherein the non-stretch fabric region is formed from a perforated fabric.

12. The lumbar support leggings of claim 8, wherein the leg openings include leg fabric extending therefrom to form legs of the leggings, wherein the leggings are configured in one of shorts, capris and pants.

13. A method for supporting a lower lumbar back of a user, comprising:
donning lumbar support leggings, the lumbar support leggings having leg openings receiving a user's legs and an open-ended torso region formed integrally with the leg openings, the open-ended torso region fitted about at least a portion of a user's torso, the open-ended torso region having a front panel formed from a flexible material with at least one side, a non-stretch fabric region configured to be disposed adjacent a user's lower lumber spine, and at least one strap extending from each side of the non-stretch fabric region, each strap configured to removably attach to each side of the open-ended torso region;
pulling on each of the straps to cause the non-stretch fabric region to apply a selected pressure to the lower lumber back of the user; and
securing the straps to the hook receiving fabric to maintain the selected pressure.

14. The method of claim 13, wherein said at least one side of the front panel has a hook receiving fabric and said strap attached to the non-stretch fabric region removably attaches to said hook receiving fabric.

15. The method of claim 13, wherein the at least one strap is at least two straps extending from each side of the non-stretch fabric region.

16. The method of claim 14, wherein the at least two straps overlap at least where the straps are attached to the non-stretch fabric region.

17. The method of claim 13, wherein the at least one strap is formed from an elastic material and the selected pressure is obtained by extending the elastic material a suitable length.
